(19) United States
(12) Plant Patent Application Publication
(10) Pub. No.: US 2009/0229022 P1
(43) Pub. Date: Sep. 10, 2009

(54) Botanical designation rosa hybrida variety denomination 'Poulpar048'

(50) ;

(76) Inventor: Mogens N. Olesen, Fredensborg (DK)

Correspondence Address: POULSEN ROSER PACIFIC, INC., 285 LIBERTY STREET NE, SUITE 370, PMB 153, SALEM, OR, 97301, US

(21) Appl. No.: 12/004,944

(22) Filed: Dec. 20, 2007

(51) Int. Cl. A01H 5/00

(52) U.S. Cl. PLT/122

(57) ABSTRACT

A new miniature rose plant that has abundant, red flowers and attractive foliage. The variety successfully propagates from softwood cuttings and is suitable for year-round production in commercial glasshouses. This new and distinct variety has shown to be uniform and stable in the resulting generations from asexual propagation.

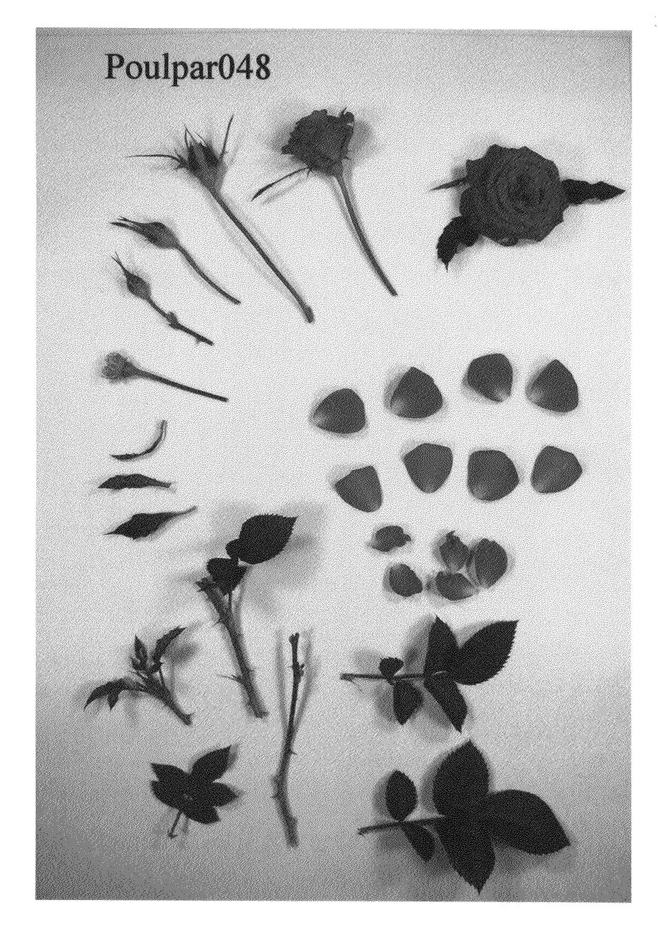

### BOTANICAL DESIGNATION

Rosa hybrida

### VARIETY DENOMINATION

‘Poulpar048’

### SUMMARY OF THE INVENTION

The present discovery constitutes a new and distinct variety of miniature rose plant which was discovered in a cultivated area. The new rose variety resulted from a naturally occurring mutation of unknown causation on a branch of ‘Poulpar029’, U.S. Plant Pat. No. 16,148. The resulting mutation was selected and evaluations were conducted on the resulting rose plants in a controlled environment.

The rose plant of the present discovery has a unique combination of characteristics which are outstanding in the new variety and which distinguish it from the original rose ‘Poulpar029’ as well as all other varieties which we are aware of. ‘Poulpar048’ tends to branch better and has larger flowers than ‘Poulpar029’.

The criteria used to evaluate the claimed plant were selected in order to introduce a distinct variety for garden use with unique qualities, such as:
1. Uniform and abundant red flowers;
2. Vigorous, but compact growth when propagated on its own roots;
3. Exceptional disease resistance; and
4. Durable foliage and long lasting flowers.

This combination of qualities is not present in previously available commercial cultivars of this type, known to the inventor, and distinguish ‘Poulpar048’ from all other varieties of which we are aware.

Asexual reproduction of ‘Poulpar048’ by vegetative cuttings was first done by Mogens N. Olesen in Fredensborg, Denmark, in July of 2001. This initial and other subsequent propagations have demonstrated that the characteristics of ‘Poulpar048’ are stable and reproduced true to type in successive generations of asexual reproduction.

### BRIEF DESCRIPTION OF THE DRAWING

The accompanying color illustrations show as true as is reasonably possible to obtain in color photographs of this type, the typical characteristics of the buds, flowers, leaves, and stems of ‘Poulpar048’.

### DETAILED DESCRIPTION OF THE VARIETY

The following is a description of ‘Poulpar048’, as observed in its growth in a glasshouse in Fredensborg, Denmark. Observed plants are 3 months of age and were cultivated in 10.5 cm pots. Color references are made using The Royal Horticultural Society (London, England) Colour Chart, 2001, except where common terms of color are used.

For a comparison, two physical characteristics of the rose variety ‘Poulpar029’, U.S. Plant Pat. No. 16,148. are compared to ‘Poulpar048’ in Chart 1.

CHART 1

| | ‘Poulpar048’ | ‘Poulpar029’ |
|---|---|---|
| Petalage: | 30 to 35 petals | 30 petals |
| Flower Diameter: | 45 to 50 mm | 35 mm |

Flower and flower bud:
   Blooming habit.—Continuous and very early.
   Flower bud.—Size: Upon opening, 20 to 25 mm in length from base of receptacle to end of bud. 7 to 9 mm in diameter. Bud form: Ovate. Bud color: As sepals unfold, petals are Red Group 53A Sepals: Upper Surface: Color: Yellow-Green Group 147A at the apex and Yellow-Green Group 146C at the base. Anthocyanic pigments the color of Greyed-Purple Group 183A are moderately expressed. Texture: Smooth. Lower Surface: Color: Yellow-Green Group 146B. Texture: Smooth. Shape: Apex: Cirrhose. Base: Flat at union with receptacle. Margins: Margins have medium foliaceous appendages on three of the five sepals. Size: 22 to 45 mm long by 4 to 10 mm wide.
   Receptacle.—Surface Texture: Smooth. Shape: Urn-shaped. Size: 7 mm in height by 7 mm wide on average. Color: Yellow Green Group 144A. Anthocyanin: None observed. Pedicel: Surface: Slightly rough with few stipitate glands. Length: 15 to 50 mm average. Diameter: 2 to 2.5 mm. Color: Yellow-Green Group 144A. Strength: Medium strength. Borne: Singly.
   Flower bloom.—Fragrance: Light floral. Duration: As a pot plant, flowers last from to 20 to 28 days. Size: Average flower diameter is 45 to 50 mm when open. Flowers are normally 25 mm in depth. Form: General shape is a miniature hybrid tea, high centered with tightly closed flower center. Shape of flower, side view: After opening, the upper portion is a flattened convex. The lower portion is concave.
   Petalage.—There are normally 30 to 35 petals under normal conditions. 3 to 5 are petaloids.
   Color.—Upon opening, petals: Outermost petals: Upper Surface: Red Group 53A. Lower Surface: Red Group 53C. Innermost petals: Upper Surface: Red Group 53A. Lower Surface: Red Group 53C. Upon opening, basal petal spots: Upper Surface: Yellow-Green Group 150D. Lower Surface: Yellow-Green Group 150D. After opening, petals: Outermost and innermost petals: Upper Surface: Red Group 46A. Lower Surface: Red Group 53C. After opening, basal petal spots: Upper Surface: Yellow-Green Group 150D. Lower Surface: Yellow-Green Group 150D.
   General tonality.—Red Group 46A until the 10^{th} to 14^{th} day. As flower further matures, tonality becomes Red Group 46B.
   Petals.—Petal Reflex: Strong. Petal Margin: Entire and uniform. With weak undulations of margin. Shape: Generally broadly elliptic. Base: Acute. Apex: Rounded. Occasionally petal apex develops shallow point. Size: On average, 20 mm in length by 20 mm wide. Thickness: Average. Texture: Smooth.
   Petaloids.—Quantity: Normally, there are 3 to 5 Size: 19 mm long; 13 mm wide. Shape: Apex is rounded. Base is acute. Generally petaloids are irregularly shaped and asymmetric. Petaloid Color: Upper Surface: Red Group 46A. Lower Surface: Red Group 53C. Basal petal spots: Upper Surface: Yellow-Green Group 150D. Lower Surface: Yellow-Green Group 150D.
   Reproductive organs.—Pollen: None observed.
   Anthers.—Size: 3 mm long. Color: Yellow Group 10C. Quantity: 55 to 60. Filaments: Color: Yellow Group 8A with intonations of Orange-Red Group 32C. Length: 7 mm. Pistils: Length: 5 mm long. Quantity: 35 to 40. Stigmas: Level relative to the length of the filaments and the height of the anthers. Color: Greyed-Yellow Group 160C. Styles: Color: Yellow-White Group 158B. Seed formation: Not observed.
Plant:
   Plant growth.—Vigorous, compact, upright to bushy. When grown as a 10.5 cm pot plant on its own roots, the average height of the plant itself is 20 cm and the average width is 16 cm.
   Stems.—Color: Young wood: Yellow-Green Group 144A. Older wood: Yellow-Green Group 146A. Internodal distance: Normally 26 mm on mature wood. Length of stems: On average, 15 cm from the base of the stem to the flowering portion. Surface Texture: Young wood: Smooth. Older wood: Smooth.
   Prickles.—Incidence: 9 prickles per 10 cm of stem. Size: Average length: 7 mm. Color: Juvenile and mature prickles are Greyed-Orange Group 174C. Shape: Concave.
   Plant foliage.—Normal number of leaflets on normal leaves in middle of the stem: 5 leaflets. Compound Leaf size: 80 mm (l)×60 mm (w) Quantity: 3 to 4 leaves per 10 cm of stem. Juvenile foliage coloration: Upper Leaf Surface: Yellow-Green Group 147A. Lower Leaf Surface: Yellow-Green Group 147B. Mature foliage coloration: Upper Leaf Surface: Yellow-Green Group 147A. Lower Leaf Surface: Yellow-Green Group 148B. Anthocyanin intonation: On plants grown under high light conditions, leaf margins on developing leaves may exhibit strong anthocyanic intonations of Greyed-Orange Group 177A.
   Plant leaves and leaflets.—Stipules: Size: 7 mm long. Shape: Linear, slightly broad based with outward extending apecies. Margins: Finely serrated. Color: Yellow-Green Group 144A. Petiole: Length: Normally 20 mm. Diameter: 1 mm. Color: Yellow-Green Group 146B with intonations of Greyed-Orange Group 176A on upper surface. Lower surface is Yellow-Green Group 146B. Underneath: Small prickles observed. Rachis: Size: Normally 20 mm. Color: Yellow-Green Group 146B with intonations of Greyed-Orange Group 176A on upper surface. Lower surface is Yellow-Green Group 146B. Lower Surface: Small prickles. Leaflet: Size: 40 mm in length by 25 mm wide. Margin: Serrate. General Shape: Elliptical. Apex Shape: Acute. Base Shape: Round. Texture: Smooth. Arrangement: Odd pinnate. Venation: Reticulate. Leaf Gloss: Not glossy.
   Disease resistance.—Above average resistance to powdery and downy mildew, black spot, and Botrytis under normal growing conditions in Fredensborg, Denmark.

1. A new and distinct variety of rose plant of the miniature class named ‘Poulpar048’, substantially as illustrated and described herein, due to its abundant, red flowers, vigorous growth, compact habit, suitability for production from softwood cuttings in pots, and durable flowers and foliage that make the variety suitable for distribution in the floral industry.
